(19) United States
(12) Patent Application Publication
(10) Pub. No.: US 2022/0008668 A1
(43) Pub. Date: Jan. 13, 2022

(54) COMPRESSOR SYSTEMS FOR BAG VALVE MASKS

(71) Applicant: SV SWISSVORTEX AG, Zurich (CH)

(72) Inventors: Andrea GUIDOTTI, Zurich (CH); Mattia ARDUINI, Chevry (FR); Pietro GOZZOLI, Zurich (CH); Luca VICENTINI, Zurich (CH)

(73) Assignee: SV SWISSVORTEX AG, Zurich (CH)

(21) Appl. No.: 17/374,026

(22) Filed: Jul. 13, 2021

Related U.S. Application Data
(60) Provisional application No. 63/051,131, filed on Jul. 13, 2020.

(51) Int. Cl. A61M 16/00

(52) U.S. Cl. CPC A61M 16/0084; A61M 16/0063

(57) ABSTRACT

A mechanical compressor system is provided for actuating a bag of a bag valve mask. Rotatable track supports are shaped so as to define respective non-circular closed-loop tracks. Each of one or more mechanical compressors includes two pins that protrude from two sides of the mechanical compressor and engage one of the closed-loop tracks. The mechanical compressors are slidingly coupled to one or more vertical sliders configured to limit the movement of the mechanical compressors to a vertical translational pattern. A bag-coupler is configured to couple the bag to the mechanical compressor system in contact with the mechanical compressors. A motor or a manual crank is arranged to rotate the rotatable track supports, such that the pins move along the closed-loop tracks, causing vertical motion of the mechanical compressors along the one or more vertical sliders, which controls a volume of the bag. Other embodiments are also described.

### CROSS-REFERENCE TO RELATED APPLICATIONS

The present application claims the benefit of U.S. Provisional Application 63/051,131, filed Jul. 13, 2020, which is assigned to the assignee of the present application and incorporated herein by reference.

### FIELD OF THE APPLICATION

The present invention relates generally to medical respirators, and in particular to medical respirators that employ a standard self-inflating bag valve mask.

### BACKGROUND OF THE APPLICATION

The regulated exchange of air between the environment and the body is essential for human life. When this exchange is irregular, reduced or interrupted, the body does not receive the right amount of oxygen. These circumstances require the intervention of an external device to ensure the exchange. Such a device should be able to recreate specific respiratory patterns and frequencies depending on the needs of the patients and the status of the patient's respiratory pathology. During pandemics, such as the COVID-19 pandemic, the demand for ventilators may exceed their availability, leading to the urgent need for simple and low-cost ventilators.

Conventional bag valve masks, also known as manual resuscitators, are hand-held devices commonly used to provide positive pressure ventilation to patients who are not breathing or not breathing adequately. The actuation of conventional bag valve masks requires the manual compression of a bag with the hand, thus requiring constant assistance by a healthcare professional to ensure a proper oxygen supply to the patient. Systems have been developed that automate the compression of the bag. These systems use the standard bag valve mask and are sometimes based on the mechanical compression of the bag by two arms actuated by stepper motors, relying on the compliance of the bag valve mask to relax the system. Other solutions use controlled pressure created by pumping air inside a sealed box in which the bag valve mask is placed.

These solutions only have limited potential for patient specific respiratory patterns as the movement of the mechanical compression steppers is difficult to control precisely, especially when requiring changing rotational velocities and the inflow of air can also be controlled only up to a certain point.

### SUMMARY OF THE APPLICATION

Embodiments of the present invention provide a mechanical compressor system configured to surround a bag of a bag valve mask, which is typically conventional, and to control the inflation and deflation of bag to create specific air flow patterns, for assisting a patient's breathing when the patient cannot breathe autonomously. When connected to the respiratory system of a patient, the actuation exerted by mechanical compressor system on bag results in a respiratory pattern without the need for constant assistance by a medical professional. Mechanical compressor system can be utilized as a replacement for respiratory ventilators in the case of a shortage or inaccessibility, in order to treat urgent respiratory conditions. By creating different respiratory patterns, the air transmission to the patient is better controlled because of the larger number of rotational tracks which have different paths that create different respiratory curves responsively the specific patient's respiratory needs. In addition, the circular pattern of the motor or the manual crank described below enables the respiratory curve to be further tailored to the patient's needs. This system adaptability is less likely to harm the patient less than other, less modular and adaptable devices known in the art.

Mechanical compressor system controls the oxygen inspiration and expiration from bag to modulate the air supply to the patient following a specific pattern, which can vary from case to case. Mechanical compressor system creates a perfectly reproducible movement between each cycle, in order to guarantee the desired oxygen inhaling and exhaling to arrive to the patient's lungs. The system is reliable over many cycles to accommodate the high number of repetitions needed throughout the treatment of the patient. The system provides customizable compression of bag.

There is therefore provided, in accordance with an application of the present invention, a mechanical compressor system for actuating a bag of a bag valve mask, the mechanical compressor system including:

one or more rotatable track supports, which are shaped so as to define respective non-circular closed-loop tracks;

one or more mechanical compressors, each of which includes two pins that protrude from two sides of the mechanical compressor, wherein the mechanical compressor system is configured such that each of the pins engages one of the closed-loop tracks;

one or more vertical sliders, to which the one or more mechanical compressors are slidingly coupled, and which are configured to limit the movement of the one or more mechanical compressors to a vertical translational pattern;

a bag-coupler, which is configured to couple the bag to the mechanical compressor system in contact with the one or more mechanical compressors; and

a motor or a manual crank, which is arranged to rotate the one or more rotatable track supports, such that the pins move along the closed-loop tracks, causing vertical motion of the one or more mechanical compressors along the one or more vertical sliders, which controls a volume of the bag.

For some applications, the mechanical compressor system further includes a watertight tank for controlling a temperature of air in the bag.

For some applications, the motor or the manual crank is configured to vary a rotational speed of the one or more rotatable track supports over the course of a rotation cycle.

The present invention will be more fully understood from the following detailed description of embodiments thereof, taken together with the drawings, in which:

### BRIEF DESCRIPTION OF DRAWINGS

FIG. 1A is a schematic illustration of a mechanical compressor system, in accordance with an application of the present invention;

FIG. 1B is a schematic illustration of a frame of the mechanical compressor system of FIG. 1A, in accordance with an application of the present invention;

FIGS. 1C and 1D are schematic illustration of a single one of one more rotatable track supports of the mechanical compressor system of FIG. 1A in isometric front view and back view, respectively, in accordance with an application of the present invention;

FIG. 1E is a schematic illustration of a single mechanical compressors of the mechanical compressor system of FIG. 1A, in accordance with an application of the present invention;

FIGS. 1F and 1G are schematic illustrations of transmission of the rotational movement of one or more rotatable track supports of the mechanical compressor system of FIG. 1A, in accordance with an application of the present invention;

FIG. 1H is a schematic illustration of parts of a rotational cycle, in accordance with an application of the present invention;

FIGS. 2A-I are schematic illustrations showing the effect of the geometry of closed-loop tracks of the mechanical compressor system of FIG. 1A on the volume of air injected into the patient's lungs during the different stages of rotation of the closed-loop tracks, in accordance with respective applications of the present invention; and

FIGS. 3A-B are schematic illustrations of the effect of the rotational pattern of a motor or a manual crank of the mechanical compressor system of FIG. 1A, in accordance with an application of the present invention.

### DETAILED DESCRIPTION OF APPLICATIONS

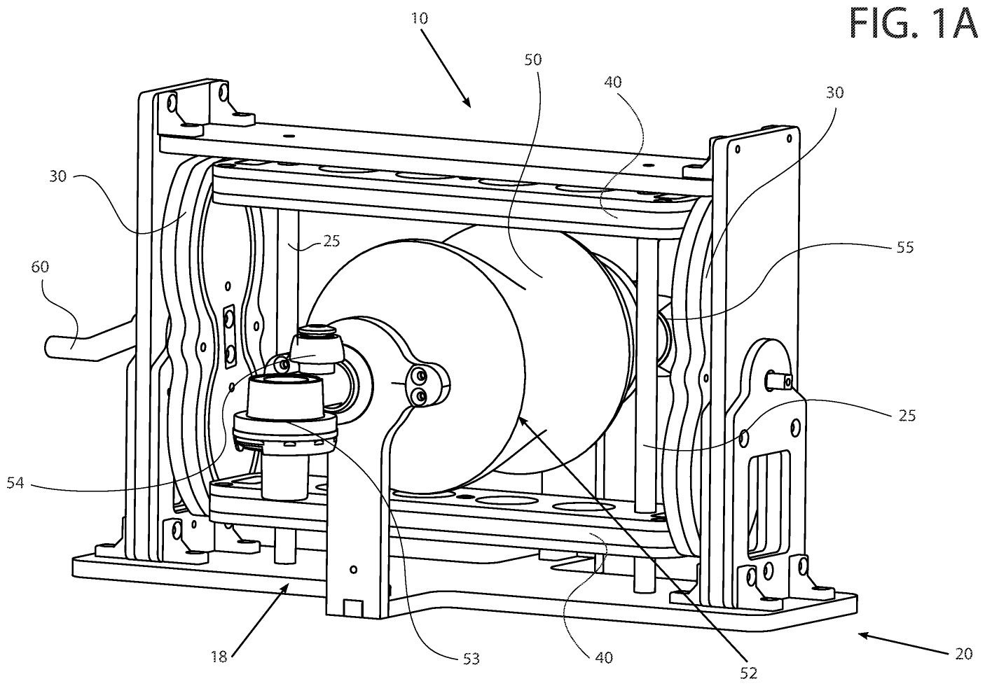

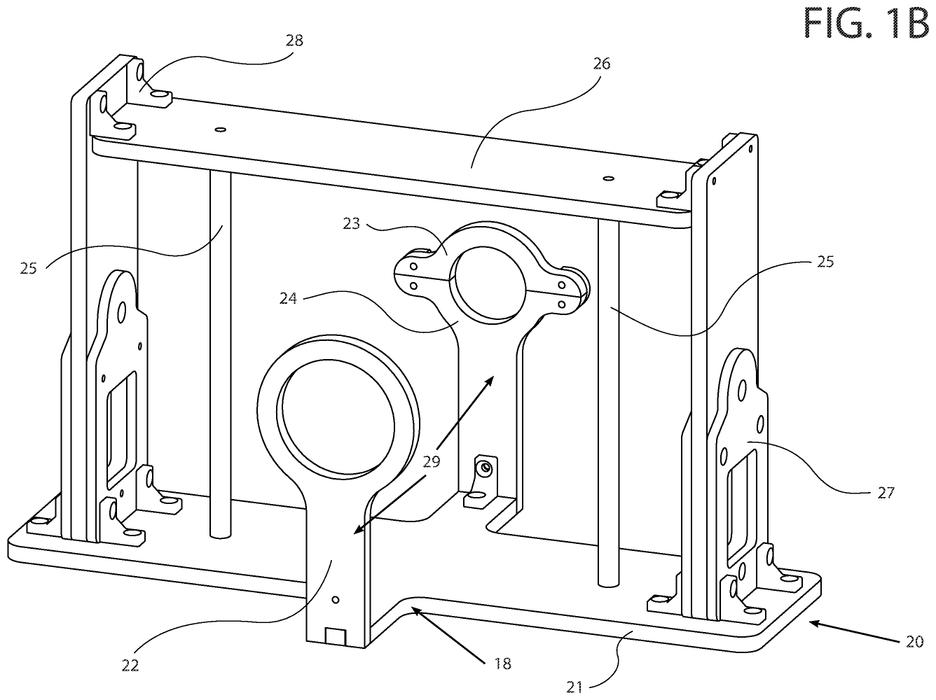

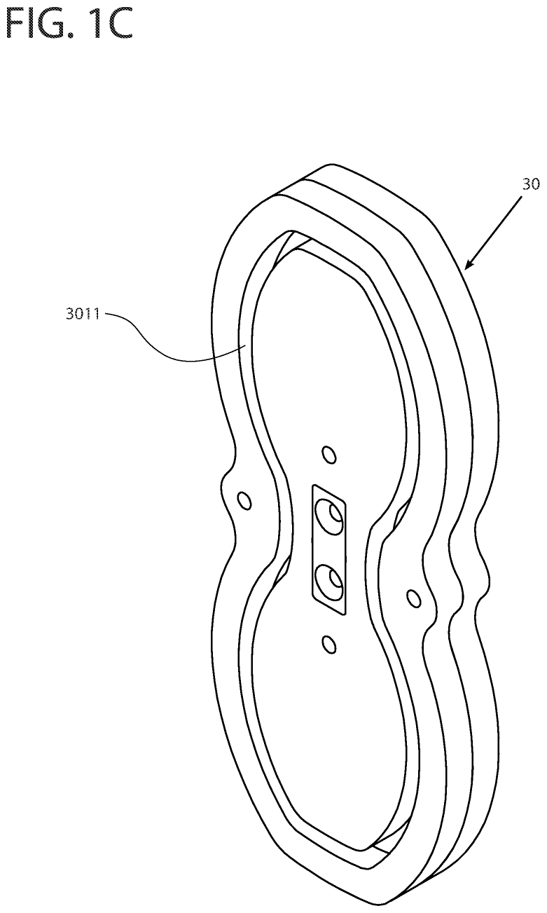

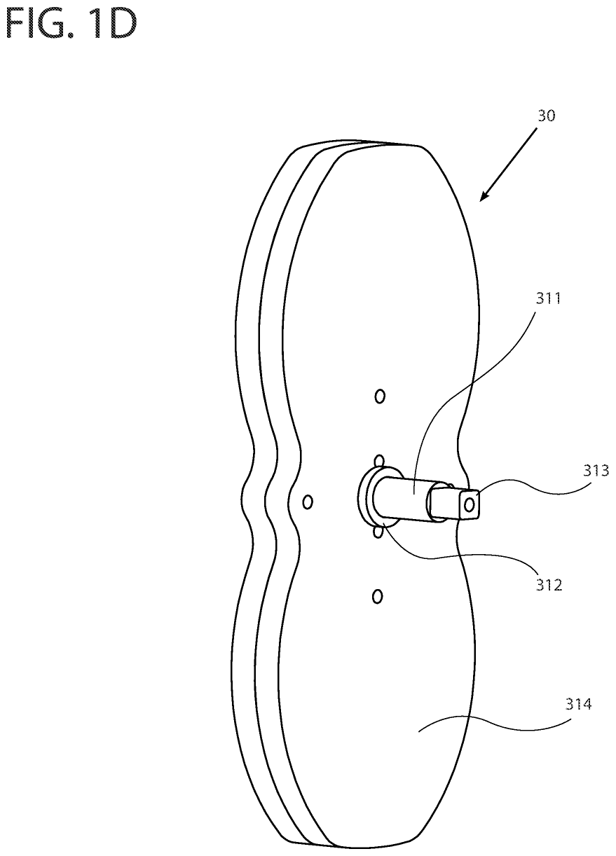

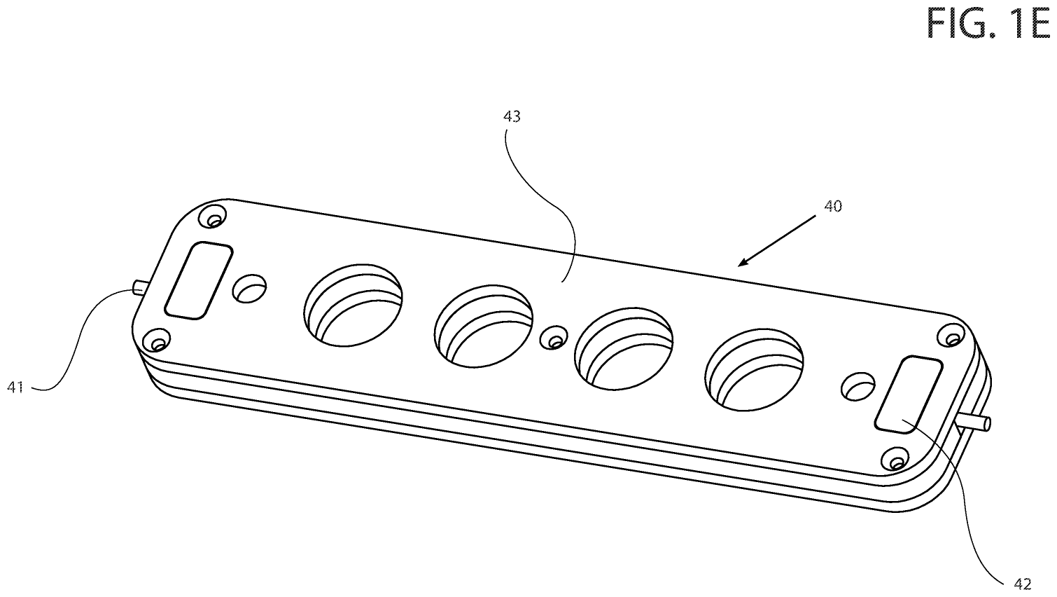

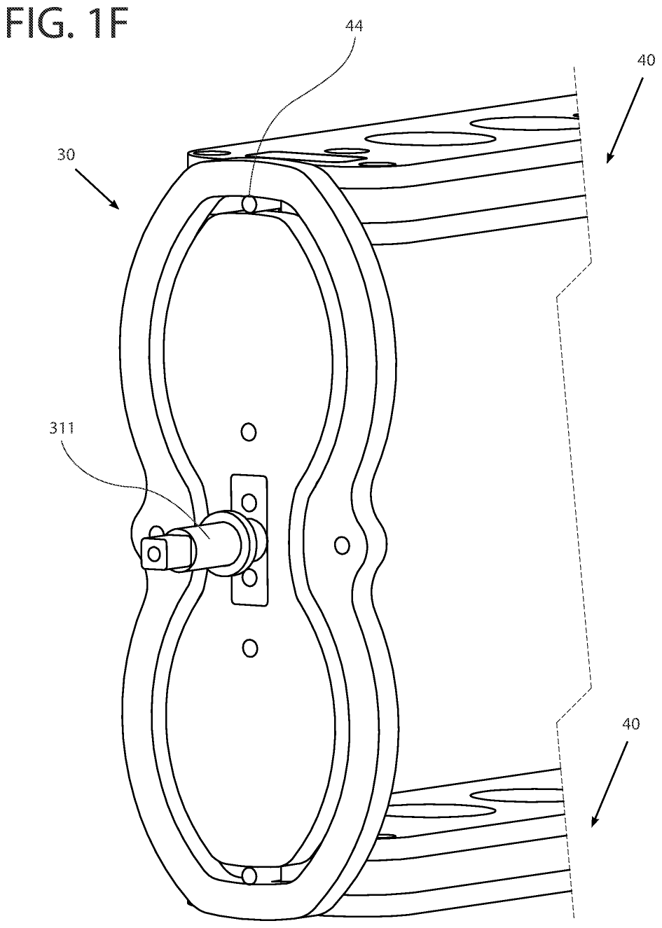

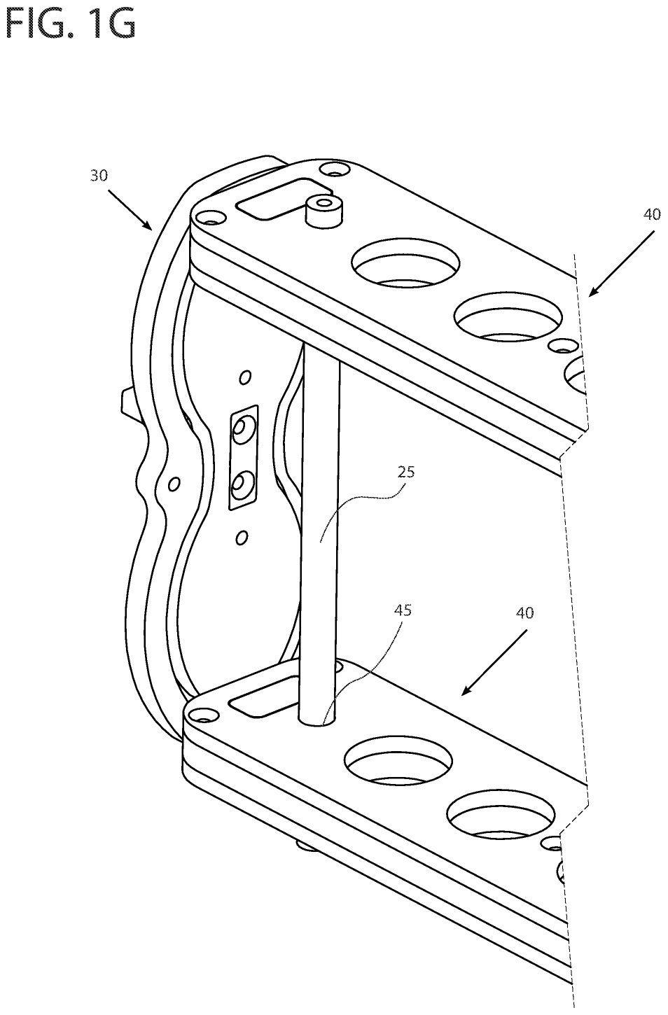

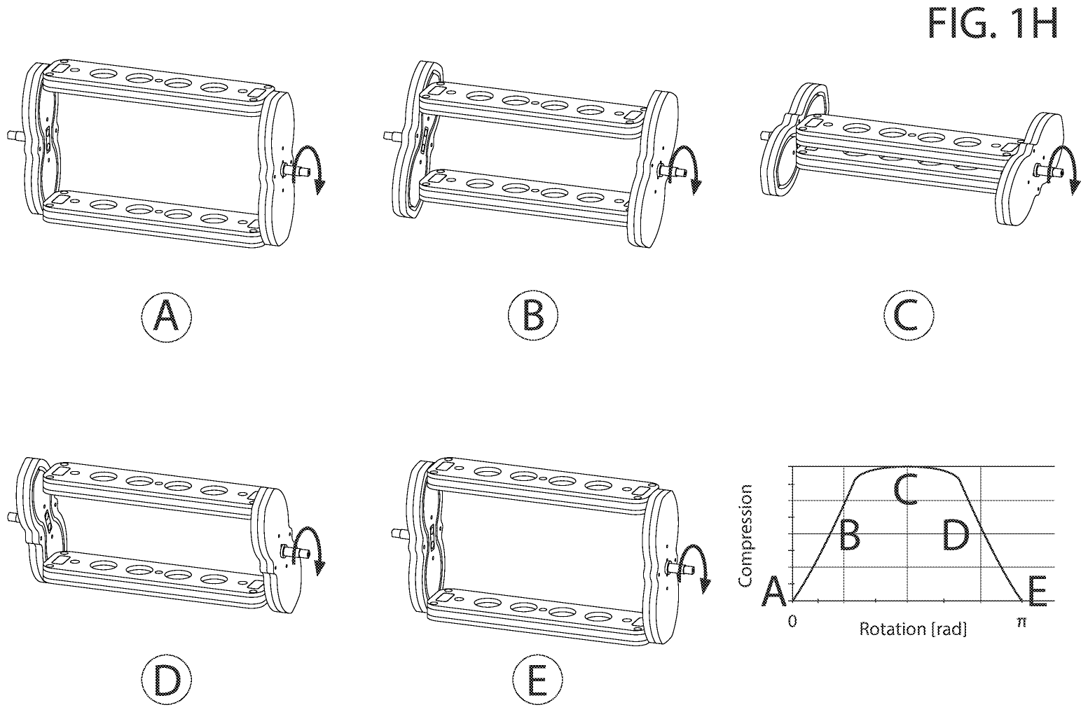

FIG. 1A is a schematic illustration of a mechanical compressor system 10, in accordance with an application of the present invention. Mechanical compressor system 10 comprises a frame 20, one or more rotatable track supports (such as exactly one, exactly two, or more than two), one or more mechanical compressors 40 (such as exactly one, exactly two, or more than two), and a motor 60 or a manual crank. FIG. 1A also shows a conventional bag valve mask 52, which comprises a conventional bag 50, surrounded by mechanical compressor system 10. Bag valve mask 52 (and bag 50 thereof) is typically not a component of mechanical compressor system 10 and is instead insertable into and removable from mechanical compressor system 10. However, mechanical compressor system 10 may alternatively comprise one or more bag valve masks 52.

Although mechanical compressor system 10 is shown in the figures as comprising exactly two rotatable track supports 30, the system may instead comprise exactly one rotatable track support 30, for example in configurations in which vertical sliders 25, described hereinbelow, and the one or more compressors 40 are very stiff, by disposing the single rotatable track support 30 on one side of the bag 50 only. Alternatively, the system may instead comprise more than two rotatable track supports 30, for example in configurations in which each of the compressors 40 is supported by two or more pins 41 and has a plus-sign shape or a polygonal shape, such as a triangular shape or a polygon with more than three sides.

As is known in the art, conventional bag valve masks typically comprise self-inflating bag 50, an expiratory valve 53, a pop-off valve 54 and a one-way air valve 55.

Reference is still made to FIG. 1A, and is additionally made to FIG. 1B, which is a schematic illustration of frame 20 of mechanical compressor system 10, in accordance with an application of the present invention. Frame 20 stabilizes and enables operation of the system. Frame 20 comprises a base plate 21, to which several fixed parts of the system are fixed, e.g., by screwing.

Frame 20 comprises a bag-coupler 29, which is configured to couple bag 50 to mechanical compressor system. 10 in contact with the one or more mechanical compressors 40. For example, bag-coupler 29 may comprise:
a distal fixation ring 22, for coupling the distal end of bag 50 to the frame; bag-coupler 29 enables the bag to be inserted by sliding it into fixation ring 22; and
two partial proximal fixation rings 23 and 24, for coupling to the proximal valve side of bag 50; for example, partial fixation rings 23 and 24 may define respective screw interfaces that allow them to be screwed together and unscrewed from each other to allow for bag 50 to be retracted or fixed in position.

Frame 20 of mechanical compressor system 10 further comprises one or more vertical sliders 25 (such as exactly one, exactly two, or more than two), which are fixed to base plate 21 of frame 20, and limit the movement of the one or more mechanical compressors 40 to a vertical translational pattern (i.e., to only allow vertical, up-and-down motion of the one or more mechanical compressors 40). The one or more vertical sliders 25 are typically shaped as respective rods, which may or may not have circular cross-sections as shown. Frame 20 optionally further comprises an upper support plate 26, to which the one or more vertical sliders 25 are fixed, in order to help stabilize the one or more sliders in the vertical position and restrain them from bending due to torque during operation of the system. Frame 20 further comprises two vertical supports 27 for supporting the one or more rotatable track supports 30. Vertical supports 27 are located on each side of the frame and enable a smooth rotation of the one or more rotatable track supports 30. For example, the components of frame 20 may be fixed together using angles 28.

Although mechanical compressor system 10 is shown in the figures as comprising exactly two vertical sliders 25, the system may instead comprise exactly one vertical slider 25, for example in configurations in which the entire system is very stiff. The number of sliders can also increase above two in order to stabilize more complex shapes of compressor 40. As an example, if compressor 40 is plus-shaped, a slider 25 can be inserted on each of the protruding parts of the shape order to stabilize the compressor 40. The same is also true for other polygonal shapes, such as triangular shapes or polygons having more than three sides.

The one or more mechanical compressors 40 may be shaped as respective plates (as shown) or respective rods, stick, laces, rubber bands, springs, or any other form of non-extendable or semi-extendable material or geometry (configurations not shown). For configurations in which mechanical compressor system 10 comprises exactly two mechanical compressors 40, such as shown in the figures, mechanical compressor system 10 is configured to provide simultaneous vertical translation of both compressors 40 toward and away from each other, along the one or more vertical sliders 25. For configurations in which mechanical compressor system 10 comprises exactly one mechanical compressor 40 (configuration not shown), mechanical compressor system 10 is configured to provide vertical translation of the single compressors 40 toward and away from a base 18 of frame 20, along the one or more vertical sliders 25. For configurations in which the system comprises exactly two vertical sliders 25, each of the one or more compressors 40 is slidably coupled to both of vertical sliders 25. One of compressors 40 is placed above bag 50 and one underneath bag 50. Compressors 40 are rigid or flexible components which, by their vertical translation, compress bag 50 by approaching each other with a specific speed. In another part of the cycle, compressors increase the distance from each other to match the diameter of bag 50, enabling bag 50 to inflate again. The motion of the one or two mechanical compressors 40 control the volume of bag 50.

Reference is now made to FIG. 1E, which is a schematic illustration of a single one of mechanical compressors 40, in accordance with an application of the present invention. Each compressor 40 comprises a rigid or semi-rigid plate 43, which is shaped to press (squeeze) or release bag 50. Each of mechanical compressors 40 comprises two pins 41 respectively protruding from two sides of the compressor (typically from the two shorter sides of the compressor). Optionally, pins 41 are fixed using ball bearings placed within two respective ball bearing holders 42 of each compressor 40.

Reference is again made to FIG. 1A, and is additionally made to FIG. 1C, which is a schematic illustration of a single one of the one or more rotatable track supports 30 in isometric front view, in accordance with an application of the present invention. Each of the one or more rotatable track supports 30 is shaped so as to define a non-circular closed-loop track 3011, which has a non-circular shape. As used in the present application, including the claims, “non-circular” means not entirely circular and includes within its scope a shape that optionally includes one or more arcuate portions. For some applications, closed-loop track 3011 is shaped as a slot (i.e., an indentation) formed within a surface of rotatable track support 30 (as shown in the figures), while for other applications, closed-loop track 3011 is shaped as a raised protrusion (i.e., a rail) from rotatable track support 30, in which case pins 41 are shaped so as to define indentations at the tips of the pins for receiving the protrusions (configuration not shown). The one or more closed-loop tracks 3011 of the one or more rotatable track supports 30 have the same shape. It is noted that although the shape of non-circular closed-loop track 3011 is shown as generally corresponding with the shape of rotatable track support 30, this is not necessarily the case, and rotatable track support 30 may have any appropriate shape, including a partly circular shape.

Mechanical compressor system 10 is configured such that each of pins 41 engages one of closed-loop tracks 3011. As a result, for configurations in which mechanical compressor system 10 comprises exactly two mechanical compressors 40, mechanical compressor system 10 is configured such that two pins 41 engage each of closed-loop tracks 3011 (such that two of the four total pins engage one of closed-loop tracks 3011, and the other two of the four total pins engage the other closed-loop track 3011). For configurations in which mechanical compressor system comprises one mechanical compressor 40, mechanical compressor system 10 is configured such that each of the two pins 41 engages one of closed-loop tracks 3011 (such that one of the two total pins engage one of closed-loop tracks 3011, and the other of the two total pins engage the other closed-loop track 3011).

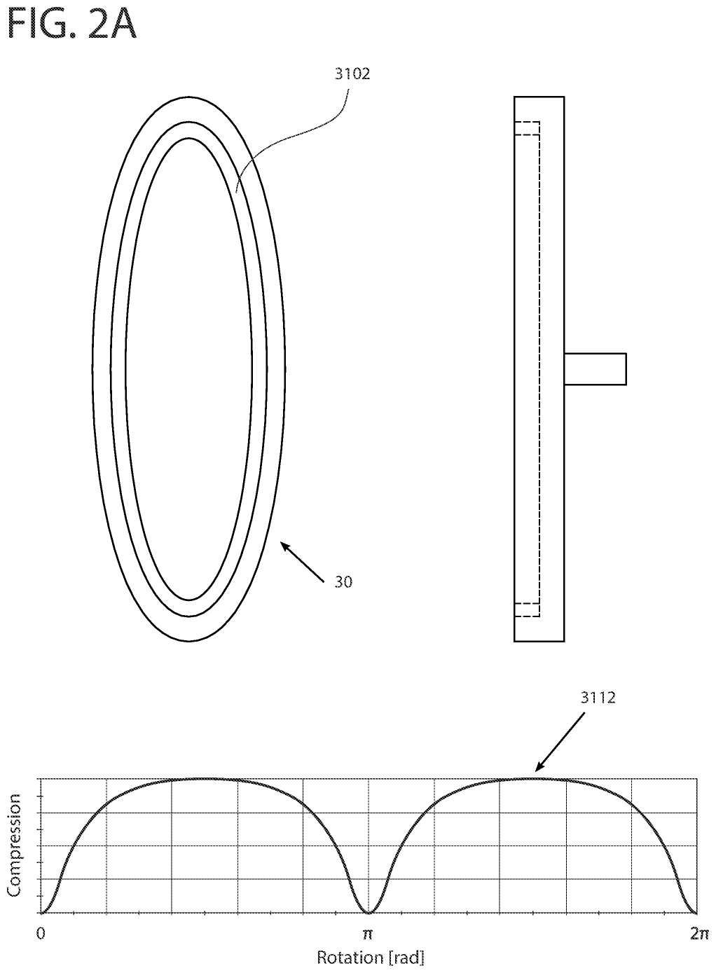

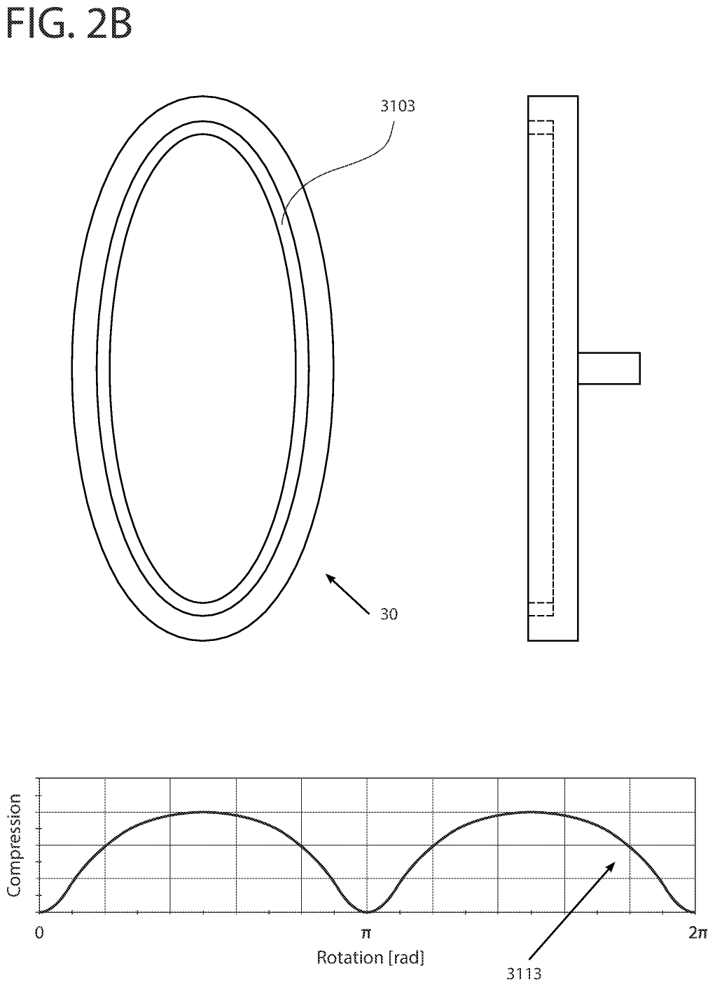

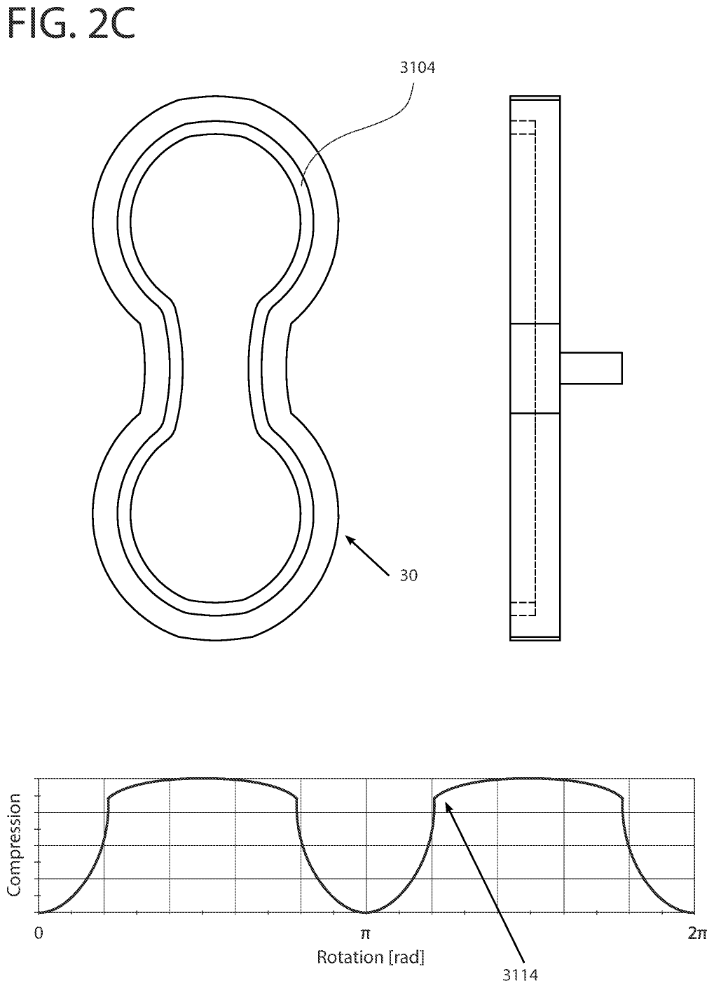

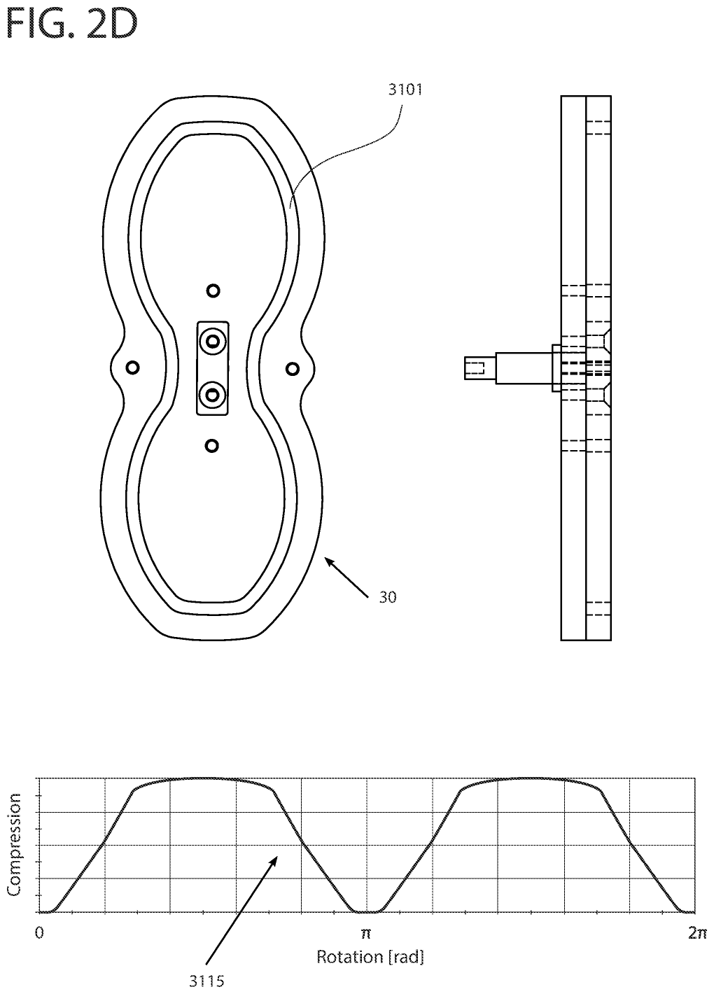

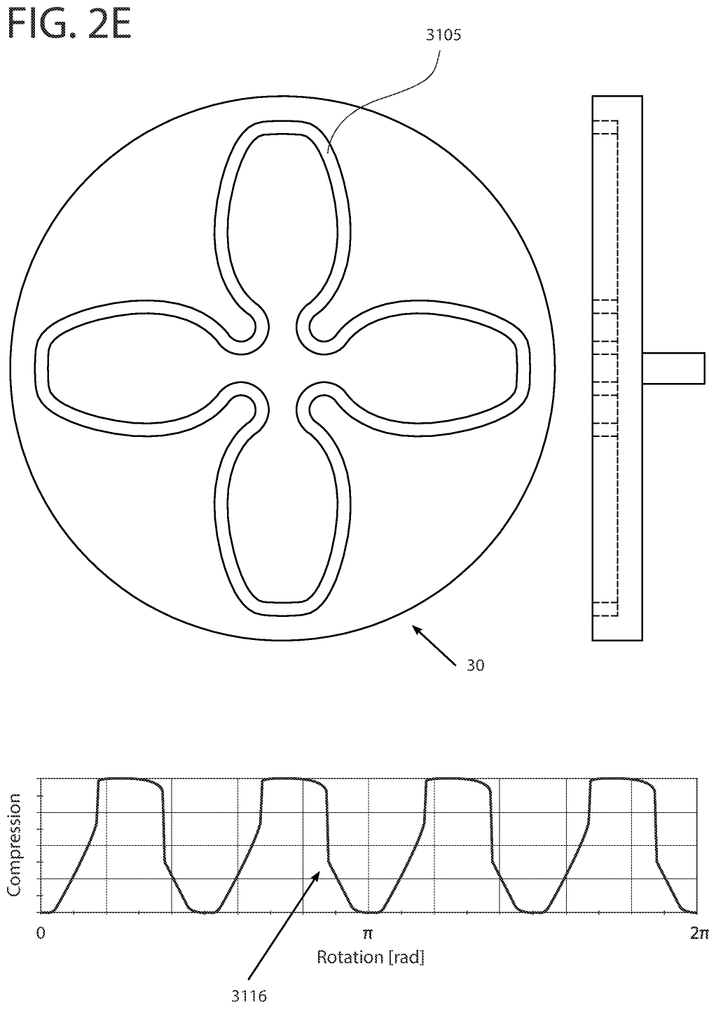

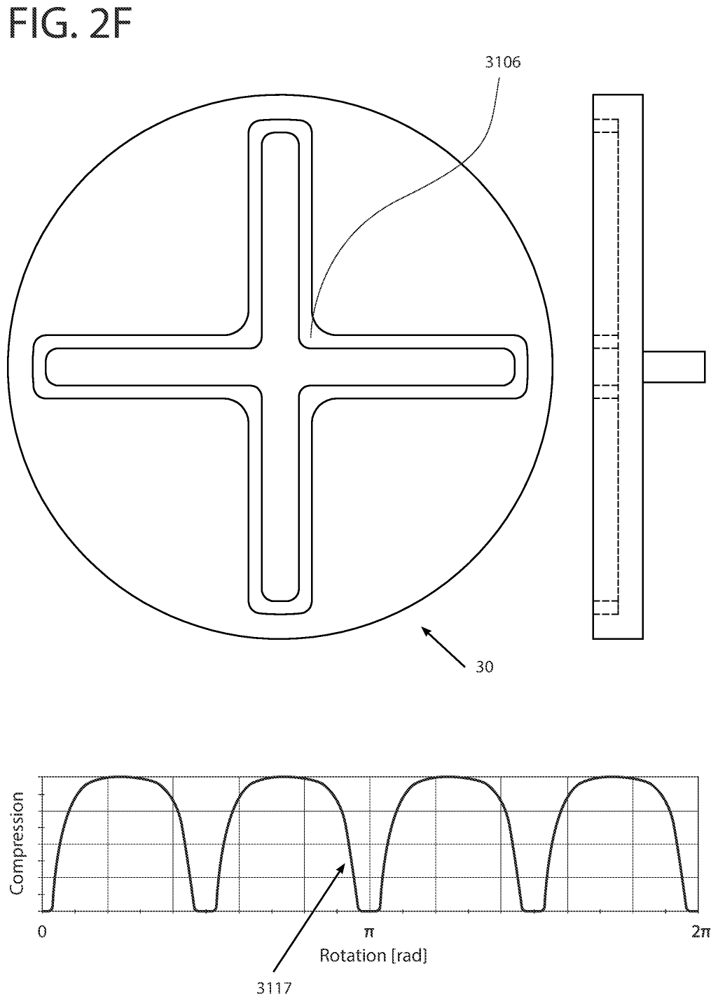

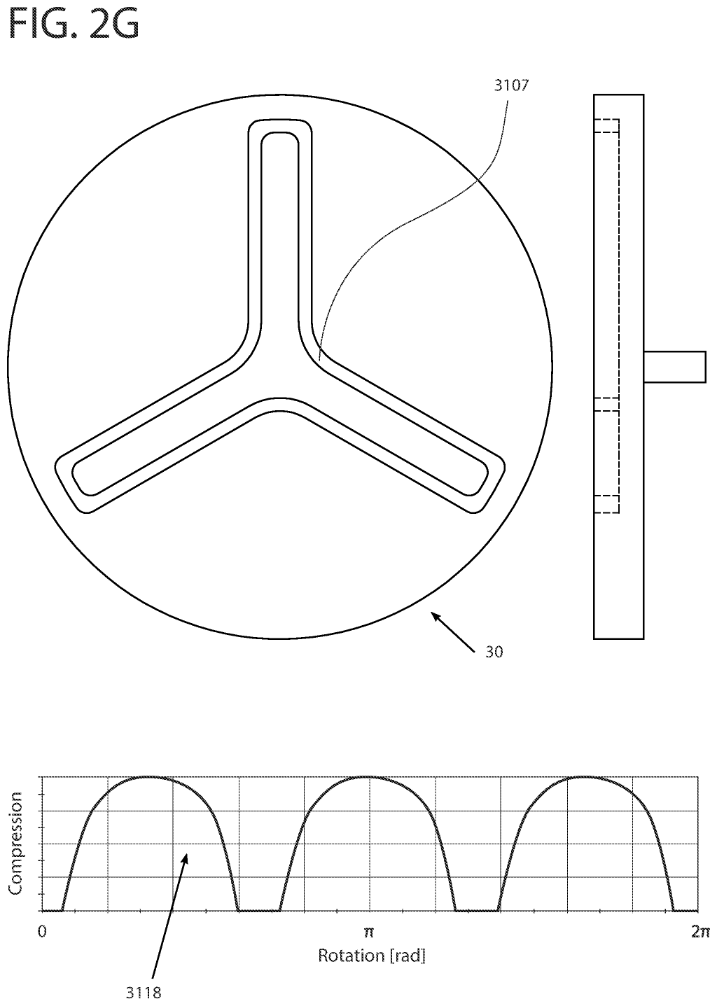

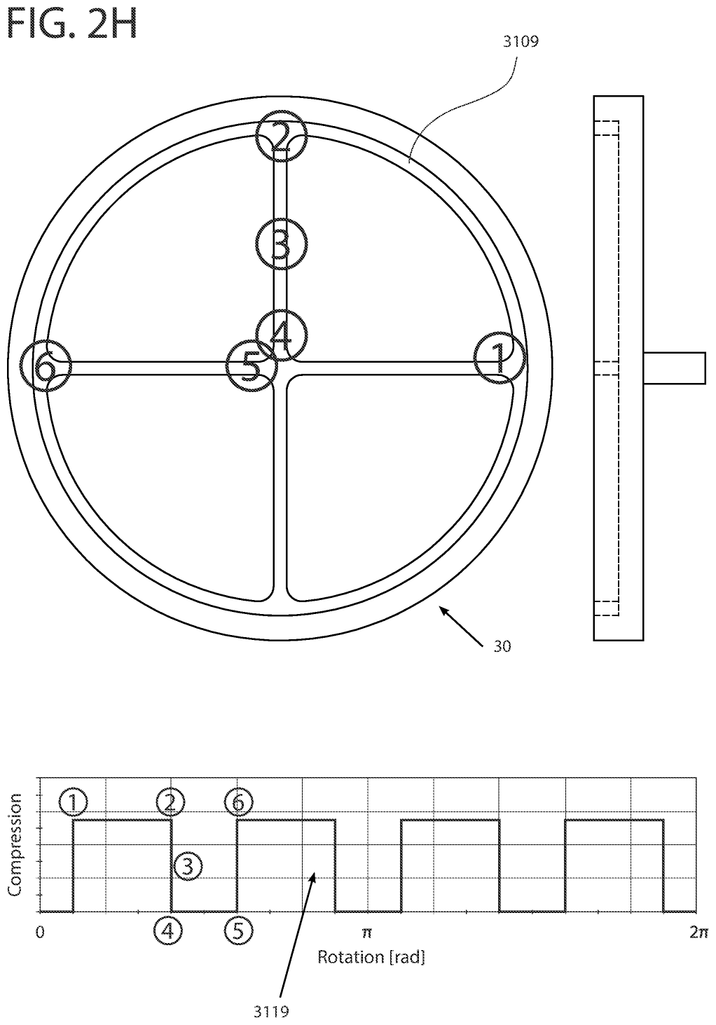

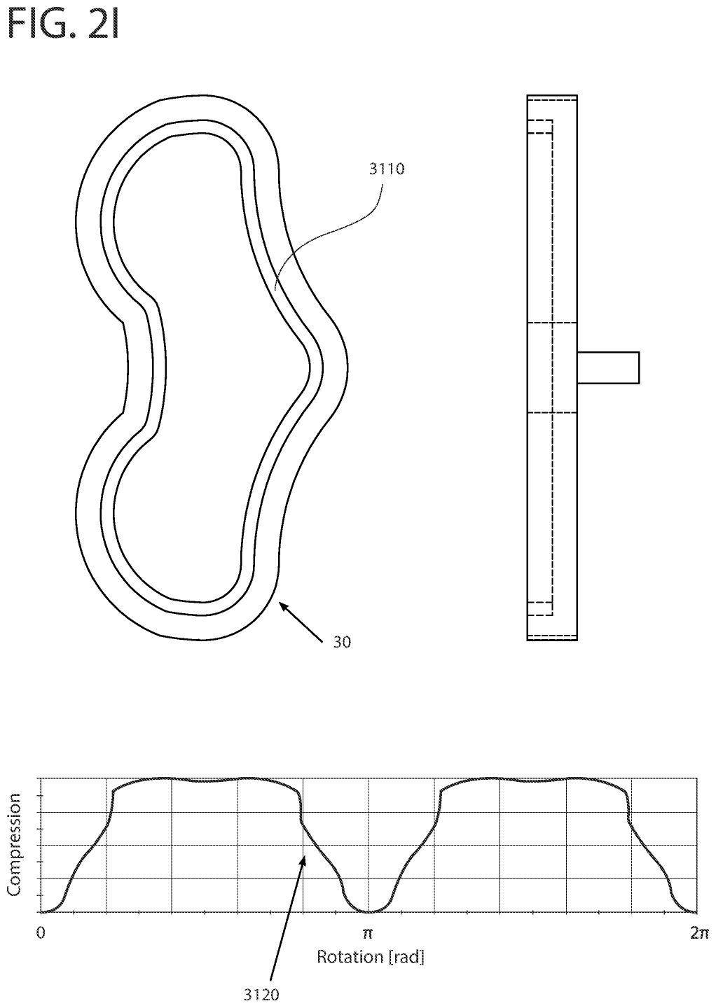

In configurations in which closed-loop track 3011 is shaped as an indentation, pins 41 are inserted into the indentation. Upon rotation of the one or more rotatable track supports 30, pins 41 move along closed-loop tracks 3011, following the shape of the closed-loop tracks 3011. As a result, the one or more mechanical compressors 40 follow the translational movement with a specific speed and according to a specific respiratory curve path, corresponding to the shape of the tracks. The speed of motor 60 or the manual crank impacts the pattern of the movement of the one or more compressors 40 and thus the respiratory pattern created for the patient. Closed-loop tracks 3011 may be provided with different shapes, each of which obtains different movement patterns of compressors 40, such as described hereinbelow with reference to FIGS. 2A-I.

The combination of the components of mechanical compressor system 10 enables the system to adapt the pattern of the respiration, as well as the time between each respiratory cycle, to each individual patient. This is achieved by the interaction of the different track shapes, the speed of motor 60 or the manual crank actuating the one or more rotatable track supports 30, and the pattern of rotation of motor 60 or the manual crank.

Frame 20 of mechanical compressor system 10 comprises base 18, which comprises base plate 21 having supports for the one or more rotatable track supports 30 on two opposite sides and two circular fixers for bag 50 on the other two sides of the base plate. The base plate is fitted with the one or more (e.g., the two) vertical sliders 25, for keeping the one or more compressors 40 on a purely translational path. Optionally, base 18 is fitted with rubber feet to ensure stability, in addition to the robustness provided through the device's own weight. Optionally, to ensure smooth rotation, the supports for the one or more rotatable track supports 30 are fixed using ball bearings. The replacement and substitution of bag 50 with another bag 50 is possible by opening the proximal fixation rings 23 and 24.

For some applications, mechanical compressor system 10 comprises a single mechanical compressor 40 only, on top of bag 50, for compressing bag 50 against base 18 of mechanical compressor system 10, with base 18 acting as second fixed compression element.

The shape of closed-loop tracks 3011 may vary in geometry, such as an elliptical shape, a figure-eight shape, a multi-lobar shape, a cross shape, or an irregular shape. Each different geometry causes the rotation of the closed-loop track 3011 to generate a specific respiratory curve path by acting on the approximation of the one or more mechanical compressors 40.

For some applications, rotatable track support 30 is shaped so as to define a plurality of closed-loop tracks 3011 (i.e., a multi-level track), to provide alternative respiratory curves. This causes the pins of the compressor to follow a non-horizontal plane on the rotatable track support 30, i.e., each of the pins follows a different path between different parts of the rotational cycle, which is includes more than one rotation of the track.

The one or more rotatable track supports 30 are activated by motor 60 or the manual crank. The speed of motor 60 or the manual crank controls the speed of rotation of the one or more rotatable track supports 30, creating different respective respiratory curve paths.

For some applications, mechanical compressor system 10 further comprises a watertight tank. The entire system can be submerged in the watertight tank with a specific water temperature that causes the air delivered to the patient's lungs to have a specific temperature.

In some applications of the invention, a method for controlling bag valve mask 52 is described. Bag 50 of bag valve mask 52 is placed between mechanical compressors 40, if two are provided, or between the single, top compressor 40 and base 18, if only a single compressor is provided. The one or more rotatable track supports 30 having the slot of varying shapes are rotated using motor 60 or the manual crank which then actuates the one or more compressors 40 which have pins 41 following the slots of the one or more rotatable track supports 30. The movement of the one or more compressors 40 is made purely translational by constraining them to move along the one or more vertical sliders 25. The shape of closed-loop tracks 3011, which the one or more compressors 40 follow, directly affects the speed and distance of the movement of the one or more compressors 40, thereby impacting the volume of air transmitted and extracted from the patient as well as the pattern of this exchange. In addition to the varying shape of closed-loop tracks 3011, the rotational scheme of motor 60 or the manual crank can be adapted further affecting the air transmission. This can, for example, be obtained by changing the rotational speed of motor 60 or the manual crank over the duration of a rotational cycle or by simply having different numbers or cycles per minute. Alternatively, motor 60 or the manual crank may operate a constant speed.

FIG. 1D is a schematic illustration of a single one of the one or more rotatable track supports 30 in isometric back view, in accordance with an application of the present invention. A horizontal rod 311 as inserted into a rotational support of the frame 20. The transmission of the rotation from motor 60 or the manual crank to the one or more rotatable track supports 30 is enabled by rod 311 and the quadratic indentation 313 on its back which can be lugged on the motor axes. Optionally, a distance ring 312 is provided to limit friction between the one or more rotatable track supports 30 and the base support.

FIGS. 1F and 1G are schematic illustrations of transmission of the rotational movement of the one or more rotatable track supports 30 being enabled through pin 41 of compressor 40 following the slot of the one or more rotatable track supports 30, in accordance with an application of the present invention. This interaction can be seen in FIG. 1F at point 44. Optionally, such as shown in FIGS. 1F and 1G, the track comprises only a single layer and lacks the back support, and the tracks are suspended in air. To ensure a perfectly translational movement of the one or more compressors 40, the one or more compressors 40 are typically constrained to follow fixed vertical sliders 25 perpendicular to the plane of the one or more compressors 40. This interaction is shown in FIG. 1G at point 45.

FIG. 1H is a schematic illustration of parts of a rotational cycle, in accordance with an application of the present invention. Position A enables bag 50 to inflate entirely. By rotation of the one or more rotatable track supports 30, Position B is attained and starts compressing bag 50. The air volume transmitted to the patient's lungs increases as bag 50 is compressed. Position C is reached at maximum compression, meaning that injected volume of air is none while the cumulative amount of air injected is maximal before starting to inflate bag 50 again during Position D and reaching full bag 50 expansion again at Position E.

FIGS. 2A-I are schematic illustrations showing the effect of the geometry of closed-loop tracks 3011 on the volume of air injected into the patient's lungs during the different stages of rotation of closed-loop tracks 3011, in accordance with respective applications of the present invention. All volume graphs are obtained over twenty-second periods with constant rotational movements at three rpm (rotations per minute). An even number of symmetry planes on closed-loop tracks 3011 enable the two or more compressors 40 to move symmetrically with respect to the middle plane of bag 50.

The geometrical shapes can vary in:
the number of axes of symmetry which influence the air volume compression capacity. The minimum number of axes of symmetry is zero if there is no symmetrical geometry, the number of axes can be one if there is symmetrical geometry, and the number of axes can reach an infinite of number of axes of symmetry, theoretically. For example, two axes are shown in FIGS. 2A-D, four in FIGS. 2E, 2F, and 2H, and one in FIGS. 2G and 2I; and/or
the number of loops of the track, as well as the number of compression and inflation points. There must be at least one compression point and one inflation point, but there can be a plurality of both points in other cases. For example, two are shown in FIGS. 2A-D, four in FIGS. 2E, 2F, and 2H, three in FIG. 2G, and one in FIG. 2I.

The center of rotation of the tracks may be, but does not have to be, in the symmetrical or Gravitational center of the track system. This configuration generates non-linear compression of bag 50 and may require a full revolution of rotatable track support 30 to provide a full respiration cycle.

In configuration in which track support 30 is symmetric, such as shown in FIGS. 2A-2D, two complete respiration cycles are provided per revolution.

FIG. 2A is a schematic illustration of a closed-loop track 3102 having a generally elliptical shape, which provides a gradual but non-constant speed of air injection and a volume curve 3112.

FIG. 2B is a schematic illustration of a closed-loop track 3103 having a generally elliptical shape with a longer minor axis than in FIG. 2A, which does not allow bag 50 to compress entirely, thereby decreasing the total column of air injected into the patient's lungs and providing a volume curve 3113.

FIG. 2C is a schematic illustration of a closed-loop track 3104 having a multi-lobed shape of cumulated transitioning volume, which provides a volume curve 3114.

FIG. 2D is a schematic illustration of a closed-loop track 3101, having a shape that causes nearly perfectly linear air volume transition to the patient through its geometry, which provides a volume curve 3115. The duration of the flat minimal and maximal curves of the volume curve 3115 are defined by the shape of the closed loop 3101.

FIG. 2E is a schematic illustration of a closed-loop track 3105 having a shape that includes four cycles, in contrast with the two cycles shown in the figures described hereinabove. This causes a doubling of the number of cycles per rotation and provides a volume curve 3116. The doubled number of cycles compared the configurations shown in the figures described hereinabove allows the mechanical compressor system to provide double the respiration cycles per complete revolution of the rotatable track support 30. It is thus possible to provide the same volume of air as would be obtained from the figures described hereinabove by rotating the rotatable track support 30 at half the frequency.

FIG. 2F is a schematic illustration of another closed-loop track 3106 having a four-cycle shape, providing a volume curve 3117.

FIG. 2G is a schematic illustration of another closed-loop track 3107, which has only one plane of symmetry, thus creating a non-symmetrical movement of compressors 40 compared to the middle plane of bag 50, thus providing a volume curve 3118. This does not allow bag 50 to be completely inflated or deflated at any point, thus reducing the range of transmitted air to the lungs. The number of cycles is fifty percent higher than with the generally elliptic shape of the closed-loop tracks shown in FIGS. 2A and 2B.

FIG. 2H is a schematic illustration of another closed-loop track 3109 having a shape that creates a binary air volume transmission, with instant inflation followed by no Transmission before instant deflation of bag 50. The compressor follows a rapid descent to the middle of the track, then follows a circular shape for a quarter of a circle during which no compression is created before allowing an instantaneous inflation of the bag, and then follows a circular path for a quarter of a circle during which the bag is fully inflated, thus providing a volume curve 3119.

FIG. 2I is a schematic illustration of another closed-loop track 3110 having a shape that shows that many closed-loop shapes are possible, including irregular shapes, which result in a different volume curve 3120.

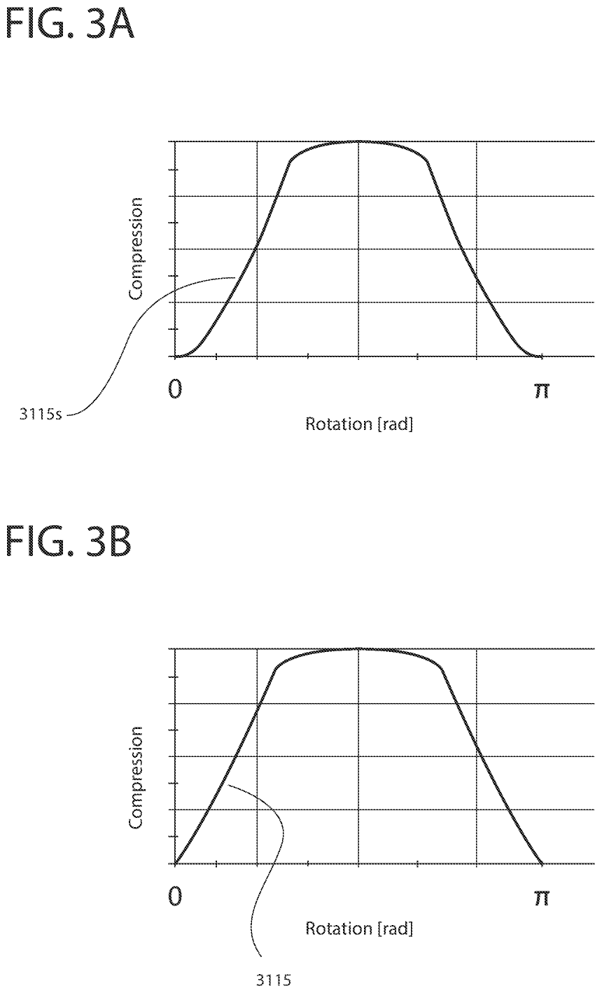

FIGS. 3A-B are schematic illustrations of the effect of the rotational pattern of motor 60 or the manual crank, in accordance with an application of the present invention. A volume curve 3115s of FIG. 3A shows the air volume transmission in the case of a sinusoidal pattern of speed compared to the constant rotational speed in a volume curve 3115 of FIG. 3B. For example, both movements may have a mean speed of three rpm.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A mechanical compressor system for actuating a bag of a bag valve mask, the mechanical compressor system comprising:
one or more rotatable track supports, which are shaped so as to define respective non-circular closed-loop tracks;
one or more mechanical compressors, each of which comprises two pins that protrude from two sides of the mechanical compressor, wherein the mechanical compressor system is configured such that each of the pins engages one of the closed-loop tracks;
one or more vertical sliders, to which the one or more mechanical compressors are slidingly coupled, and which are configured to limit the movement of the one or more mechanical compressors to a vertical translational pattern;
a bag-coupler, which is configured to couple the bag to the mechanical compressor system in contact with the one or more mechanical compressors; and
a motor or a manual crank, which is arranged to rotate the one or more rotatable track supports, such that the pins move along the closed-loop tracks, causing vertical motion of the one or more mechanical compressors along the one or more vertical sliders, which controls a volume of the bag.

2. The mechanical compressor system according to claim 1, further comprising a watertight tank for controlling a temperature of air in the bag.

3. The mechanical compressor system according to claim 1, wherein the motor or the manual crank is configured to vary a rotational speed of the one or more rotatable track supports over the course of a rotation cycle.
